(19) United States
(12) Patent Application Publication
(10) Pub. No.: US 2009/0029387 A1
(43) Pub. Date: Jan. 29, 2009

(54) METHOD FOR EFFECTIVELY MEASURING THE ACTIVITY OF CYTOTOXIC T LYMPHOCYTES IN HUMAN AND OUT-BRED ANIMALS

(75) Inventors: Sang Won Jung, Seoul (KR); Yong Soo Bae, Gyeonggi-do (KR); Choon Ju Jeon, Suwon- Si (KR)

Correspondence Address: THE RAFFERTY PATENT LAW FIRM, 1952 Gallows Road, Suite 200, Vienna, VA, 22182-3823, US

(73) Assignee: CREAGENE, INC., Seoul (KR)

(21) Appl. No.: 11/915,738

(22) PCT Filed: May 25, 2006
(86) PCT No.: PCT/KR2006/002029

§ 371 Date: Feb. 13, 2008

(30) Foreign Application Priority Data
May 27, 2005 (KR) 10-2005-0045234

(51) Int. Cl. G01N 33/566

(52) U.S. Cl. 435/7.1

(57) ABSTRACT

A method for measuring the activity of cytotoxic T lymphocytes (CTLs) includes preparing peripheral blood mononuclear cells (PBMCs) from blood of an animal; preparing mature dendritic cells by isolating monocytes from the PBMCs, differentiating the monocytes into dendritic cells for presenting an antigen molecule and pulsing dendritic cells with the antigen molecule to obtain the mature dendritic cells; preparing the CTLs as an effector cell by stimulating the PBMCs with the mature dendritic cells to activate and amplify the CTLs; preparing target cells by pulsing the PBMCs, monocytes or B cells with a cytoplasmic transduction peptide (CTP)-antigen complex generated by linking the antigen molecule of step (b) to the CTP; treating the target cells with the effector cells; and analyzing the lysis of the target cells. In addition, a kit for measuring the activity of cytotoxic T lymphocytes is provided.

### CROSS-REFERENCES TO RELATED APPLICATIONS

This is a US National Stage of International Application PCT/KR2006/002029, filed Nov. 27, 2006, which was published as International Publication WO 2006/126865 on Nov. 30, 2006. The benefit of priority is further claimed to Republic of Korea patent application KR 10-2005-0045234, filed May 27, 2005, which was registered as Republic of Korea Patent Reg. No. 10-0734525 on Jul. 3, 2007. Each application to which the benefit of priority is claimed is incorporated herein by reference in its entirety.

### INCORPORATION OF SEQUENCE LISTINGS

The accompanying Sequence Listings, including SEQ ID NO:1 through SEQ ID NO:17, which were submitted electronically in computer-readable format in the file having filename “P10073-SEQ1.txt,” 9 kB in size, are incorporated herein by reference in their entireties.

### FIELD OF THE INVENTION

The present invention relates to a method for measuring the activity of cytotoxic T lymphocytes (CTLs), and particularly, to a method for measuring the activity of CTLs induced in human beings and out-bred animals without the use of antigen-expressing recombinant viruses, and a corresponding kit therefor.

### BACKGROUND

The effect and efficacy of vaccines against cancer and other incurable diseases developed by using dendritic cells are closely related to the activity and level of cytotoxic T lymphocytes (CTLs) induced in the vaccinated subject (Unanue, 1984 Babbitt et. al., 1985 Germain, 1986 Yewdell and Bennink, 1990). In this regard, research efforts for developing cell therapy products and vaccines for preventing or treating incurable diseases have been mainly focused on CTL-inducing potential (Harding and Song, 1994 Falo et. al., 1995 Boon et. al., 1997). In particular, reports on the development of AIDS vaccines describe that the efficacy of vaccines is proportionally related to the CTL inducibility (McMichael and Rowland-Jones, 2001).

Although the efficacy of vaccines is determined depending on their CTL-inducing ability, a measurement system for CTL has not yet been established for human beings and out-bred animals such as monkeys (Piriou et. al., 2000). Currently, vaccines developed for preventing or treating incurable viral-diseases (e.g., hepatitis and AIDS) and cancer have been tested only using out-bred laboratory animals from which cell lines having matched MHC (major histocompatibility complex) had already been prepared. Therefore, the CTL-inducing potential of vaccines has not yet been analyzed effectively for out-bred animals or human beings from which cell lines having matched MHC have not been prepared beforehand. Specifically, it is indispensable to check the effective induction of immune responses in vaccinated subjects because the ability to induce CTL in human beings has been shown to differ greatly from individual to individual. However, a commercialized product for CTL measurement still has not been developed for incurable diseases such as AIDS.

Tailor-made drugs will become predominant in the 21^{st} century. For prognosis of diseases, it is necessary to measure the level of CTL induction after injection of therapeutic vaccines to patients. In addition, when vaccines for preventing incurable diseases such as cancer are administered to subjects, it is a crucial step to measure individually the activity of CTL. Accordingly, the measurement of CTL activities will be predominantly preformed in the near future.

MHC-matched target cells are essentially required to measure the activity of CTL against a specific antigen. MHC-matched cell lines usually available from in-bred animal models can be manipulated to expressing the antigen of interest and are then used as target cells for the measurement of CTL activity (Kao et. al., 2003).

However, livestock and human beings as a main subject for vaccine development are out-bred and therefore require to establish each cell line individually in order to measure CTL activity; however, such an approach is impracticable. Therefore, primary cells themselves have been used as a target cell. In such case, the vaccinia virus is used as a gene delivery system to transduce genes encoding antigens of interest into primary cells. However, the vaccinia viral vector requires Bio-Safety Level 3 (BL-3) facilities and produces varying results depending on the surrounding environment, experimental conditions and researchers, resulting in fluctuating results in experiments. In connection with this, the measurement of CTL activity has not yet become a generalized procedure even among vaccine specialists.

Throughout this application, several patents and publications are referenced and citations are provided in parentheses. The disclosure of each of these patents and publications is incorporated into this application in order to more fully describe this invention and the state of the art to which this invention pertains.

### SUMMARY

For overcoming limitations associated with conventional technologies, the present inventors have conducted intensive research to develop a novel method for measuring the activity of CTL using primary cells as target cells. As a result, we have discovered that where antigens linked to a cytoplasmic transduction peptide (CTP) previously developed by us are introduced into primary cells, target cells for measuring the activity of CTL can be prepared in a more feasible and effective manner. In addition, we have found that where the lysis of the target cells by effector cells is measured, the activity of CTL can be analyzed in a more effective and reliable manner.

Accordingly, it is an object of this invention to provide a method for measuring the activity of cytotoxic T lymphocytes (CTLs). It is another object of this invention to provide a kit for measuring the activity of CTLs. Other objects and advantages of the present invention will become apparent from the detailed description to follow taken in conjugation with the appended claims and drawings.

In one aspect of this invention, there is provided a method for measuring the activity of cytotoxic T lymphocytes (CTLs), which includes preparing peripheral blood mononuclear cells (PBMCs) from blood of an animal; preparing mature dendritic cells by isolating monocytes from the PBMCs, differentiating the monocytes into dendritic cells for presenting an antigen molecule and pulsing the dendritic cells with the antigen molecule to obtain the mature dendritic cells; preparing the CTLs as an effector cell by stimulating the PBMCs with the mature dendritic cells to activate and amplify the CTLs; preparing target cells by pulsing the PBMCs, monocytes or B cells with a cytoplasmic transduction peptide (CTP)-antigen complex generated by linking the antigen molecule of step (b) to the CTP; treating the target cells with the effector cells; and analyzing the lysis of the target cells.

For overcoming limitations of conventional technologies, the present inventors have made intensive research efforts to develop a novel method for measuring the activity of CTL using primary cells as target cells. As a result, we have discovered that where antigens linked to a cytoplasmic transduction peptide (CTP) previously developed by us are introduced into primary cells, target cells for measuring the activity of CTL can be prepared in more feasible and effective manner. In addition, we have found that where the lysis of the target cells by effector cells is measured, the activity of CTL can be analyzed in more effective and reliable manner without preparing MHC-matched cell lines and cloning recombinant viruses for transduction.

The present invention ensures target cells to be prepared by use of primary cells without establishing cell lines. In addition, the present invention enables to measure more accurately and conveniently the activity of CTL by no use of recombinant viruses for transduction and radioisotopes. Accordingly, it could be understood that the present invention is considerably useful in determining whether the immune responses are effectively induced upon vaccination, ensuring to provide a promising approach for much more reliable and convenient prognosis of diseases.

### BRIEF DESCRIPTION OF THE DRAWINGS

FIG. 1 is a genetic map of the pCTP-Td vector for expressing CTP (cytoplasmic transduction peptide)-p24 and CTP-Nef. PT7, T7 promoter; 6×His, a tag having six His residues; f1 ori, f1 replication origin; and titles of antibiotics, resistance genes to corresponding antibiotics.

FIG. 2a is a photograph representing purified CTP-p24 and CTP-Nef expressed by the pCTP-Td (Amp^{R} or Kan^{R}) vector. Lane 1, unpurified; lane 2, a flow-through fraction of Ni⁺-NTA chromatography; lanes 3-4, washing results; and lanes 5-7, elutes.

FIG. 2b is a photograph representing purified CTP-HCVcore expressed by the pET43.1 vector. M, molecular marker; lane 1, a flow-through fraction of Ni⁺-NTA chromatography; lane 2, washing results; and lanes 3-4, elutes using 50 mM and 100 mM imidazole, respectively.

FIGS. 3a-3c represent results of the Western blotting for verifying the transduction potential of CTP-p24, CTP-Nef and CTP-HCVcore, respectively. In FIG. 3c, “control” represents result of CTP-HCVcore purified from E. coli and “Ut” corresponds to untreated dendritic cells.

FIG. 3d represents results of the dot blotting for quantitating the CTP-p24 antigen transported into cells. “PTD” represents a conventional peptide transduction domain, amino acid sequence of which is YGRKKRRQRRR. The results demonstrate that the recombinant CTP-p24 antigen exhibits much higher transduction potential than PTD-p24.

FIG. 4 represents results of FACS dot-blot analyses on costimulatory factor on the surface of dendritic cells to verify the generation of mature dendritic cells.

FIG. 5 represents results of 20-day coculturing with non-adherent PBMCs and dendritic cells pulsed with CTP-p24. Panel A, proliferation of CD3⁺ T cells; panel B, percentages of CD8⁺ T cells in total T cells proliferated; panel C, levels of γ-IFN secreted in coculturing; and panel D, levels of IL-4 secreted in coculturing.

FIGS. 6a and 6b represent FACS analysis results of experiments for establishing optimal CFSE labeling process. P815 cell line (FIG. 6a) and mouse splenocytes (FIG. 6b) pulsed with CTP-p24 were labeled with a relatively high dose of CFSE (6, 12 and 20 μM); those not pulsed with antigen were labeled with a relatively low dose of CFSE (2, 4 and 6 μM). To mimic the influence of excess effector cells (used in 10-fold or 20-fold number), the labeled cells were mixed with 10-fold or 20-fold number of unlabeled splenocytes. We examined to determine whether the lysis of target cells pulsed with CTP-p24 could be distinctly detected among CFSE-labeled cells by FACS.

FIGS. 7a-7d show results of the measurements of CTL activity according to the present invention. To reveal a method for elevating the sensitivity of target cells, PBMCs pretreated with 1000 U/ml of γ-IFN and PBMCs untreated were respectively pulsed with 50 μg/ml of CTP-p24 and used as target cells for CTL analysis (FIG. 7a). In addition, to determine whether the use of monocytes isolated from PBMCs could increase the sensitivity of target cells, isolated monocytes and PBMCs were respectively pulsed with CTP-p24 and used as target cells for CTL analysis (FIG. 7b). To verify the specificity to p24, PBMCs pulsed with CTP-p24 or CTP-albumin (human serum albumin) were used as target cells for measuring CTL activity against p24 (FIG. 7c). To determine whether activities observed in the measurements of CTL activity are antigen-specific, Nef-specific CTLs were generated and their activity was measured by using PBMCs pulsed with CTP-Nef or CTP-albumin as target cells (FIG. 7d).

FIG. 8 shows results of the measurements of CTL activity according to the present invention. With increasing the number of target cells by ten-fold, the ratio of effector cells to target cells (E/T) was varied and the CTL activities were measured. PBMCs pretreated with 1000 U/ml of γ-IFN for 7 hr were pulsed with 50 μg/ml of CTP-p24. The reactions of effector and target cells were adjusted by varying the number of effector cells with a fixed number of target cells (1×10⁵ cells/well) and the CTL activities were measured.

### DETAILED DESCRIPTION

The present invention will be described in more detail hereunder:

Step (a): Preparing Peripheral Blood Mononuclear Cells (PBMCs) from Blood

PBMCs may be obtained from blood in accordance with conventional procedures known to one skilled in the art. For example, blood from subjects (e.g., AIDS patients and vaccinated persons) for the activity of CTL is collected and then diluted with PBS, if necessary. Then, the blood samples are centrifuged with density gradient medium such as ficol-paque solution or Lymphoprep™ to obtain peripheral blood mononuclear cells, PBMCs.

The term “peripheral blood mononuclear cell (PBMC)” used herein refers to any cell containing one nucleus found in peripheral blood, for example, including monocyte, T cell, B cell and NK cell. According to a preferred embodiment, peripheral blood mononuclear cells are primary cells.

In the present invention, the type of animals, a subject of CTL measurement, is not restricted, preferably out-bred animals, most preferably human.

Step (b): Preparing Mature Dendritic Cells from PBMCs

Monocytes are isolated from the PBMCs prepared from blood, differentiated into dendritic cells and then pulsed with an antigen molecule to obtain mature dendritic cells.

The PBMCs suspension obtained in step (a) is aliquoted into a suitable medium (e.g., RPMI 1640 medium) and cultured to adhere monocytes to culturing plates for isolating, followed by removing non-adherent cells. In other words, the isolation of monocytes from peripheral blood mononuclear cells is carried out by use of plastic adherency of monocytes to culturing devices for animal cells.

Thereafter, adherent cells, monocytes are cultured in a medium for dendritic cells (e.g., X-VIVO) containing cytokines, to induce the differentiation into dendritic cells. Exemplified cytokines useful in this step include IL-4 (interleukin-4) and GM-CSF (granulocyte macrophage-colony stimulating factor). The period of time for differentiation culturing is usually more than 5 days.

The immature dendritic cells obtained thus are pulsed with antigen molecules to give mature dendritic cells. The immature dendritic cells are cultured with suitable cytokines and antigens to maturate dendritic cells. Maturation factors used include a cytokine cocktail with a specific composition and monocyte-conditioned medium with non-specific composition (MCM: monocyte-conditioned medium Armin Bender et al., Journal of Immunological Methods 196:121-135 (1996)). Cytokine contained in a cytokine cocktail with a specific composition comprises TNF-α, IL-1β, IL-6, PGE2 (prostaglandin E2), IFN-γ and their mixture. Besides the cytokine cocktail and non-specific monocyte-conditioned medium, T cell factor CD40L, maturation factors such as TRANCE and stabilizing factors may be used as a sole or supplementary factor for maturation of dendritic cells. In addition, microbes-derived substances such as CpG, SAC, SEB and LPS (lipopolysaccharide) are used in this step for enhancing stimulation. The period of time for maturation culture is usually more than 1 day, more preferably, 1-3 days.

The antigen for pulsing dendritic cells in the maturation step has preferably a fusion form in which it is linked to the CTP. The CTP molecules are described in the descriptions of the present kits. The CTP delivers antigens into cytoplasm not nucleus, which permits dendritic cells to present more effectively antigens on their surface through major histocompatibility complex class I (MHC I) molecules. The descriptions of CTP molecules are also found in WO 03/097671, the teachings of which are incorporated herein by reference.

Where the present invention is intended to measure the activity of CTL against AIDS, it is preferable to pulse dendritic cells with CTP-p24 or CTP-Nef antigen. Where the present invention is intended to measure the activity of CTL against hepatitis C, it is preferable to pulse dendritic cells with CTP-HCVcore antigen.

Step (c): Amplifying and Preparing Effector Cells from PBMCs

The starting material to amplify and prepare effector cells is PBMCs derived from blood or a CTL-containing cell population of PBMCs. For example, the effector cells may be prepared by use of non-adherent PBMCs removed in step (b) for preparing dendritic cells. The non-adherent PBMCs and mature dendritic cells are cocultured to simulate PBMCs to amplify CTLs as effector cells. For coculturing, the ratio of mature dendritic cells to PBMCs is 1:4-1:20, preferably 1:5-1:15, more preferably 1:8-1:12, most preferably 1:10. The period of time for stimulation to prepare effector cells is preferably 1-4 weeks, more preferably 2-3 weeks.

Step (d): Preparing Target Cells

Target cells are prepared using PBMCs, monocytes or B cells derived from animal blood, preferably, PBMCs. The PBMC used to prepare target cells is fresh, freezing-thawed, or stimulated one with suitable cytokines (e.g., IFN-γ, TNF-α, IL-1β, IL-6, PGE2, IL-4, GM-CSF and Con-A). Preferably, the PBMCs are a primary cell. The PBMCs as target cells are preferably autologous to PBMCs used to prepare effector cells. PBMCs, monocytes or B cells for preparing target cells are isolated from animal, preferably out-bred animal, most preferably human.

The PBMCs are pulsed with a CTP-antigen complex generated by linking the antigen molecule of step (b) to the CTP for a period of time (preferably 10-40 hr, more preferably 10-30 hr, most preferably 15-20 hr). The descriptions for the CTP follow those of the present kits as described hereunder.

One of the features of the present invention is to use primary cells as target cells. Such successful application of primary cells is mainly due to the use of the CTP-antigen complex as antigens for pulsing cells. The CTP delivers antigens into cytoplasm not nucleus, which permits target cells to present more effectively antigens on their surface through major histocompatibility complex class I (MHC I) molecules. In this respect, it could be appreciated that target cells having surface-presented antigens susceptible to the activity of CTLs are more conveniently generated in accordance with this invention. The descriptions of CTP molecules are also found in WO 03/097671, the teachings of which are incorporated herein by reference.

Where the present invention is intended to measure the activity of CTL against AIDS, it is preferable that PBMCs are pulsed with CTP-p24 or CTP-Nef antigen to generate target cells. Where the present invention is intended to measure the activity of CTL against hepatitis C, it is preferable that PBMCs are pulsed with CTP-HCVcore antigen to generate target cells.

Target cells prepared thus are labeled for making it more facile to performing the analysis of step (f). Exemplified labels used include DiOC (3,3′-dioctadecycloxacarbocyanine), Calcian-AM (acetoxymethyl ester of calcian), CM-FDA, CM-TMR and CFSE (carboxyfluorescein diacetate succinimidyl ester), most preferably CFSE. For more accurate analysis, cells untreated with CTP-antigen are also labeled as internal control.

For instance, where target cells are labeled with CFSE, antigen-pulsed target cells are labeled with 12-30 μM, preferably 15-25 μM, more preferably 18-23 μM, most preferably 20 μM CFSE. In addition, control cells untreated with antigens are labeled with 2-10 μM, preferably 3-8 μM, more preferably 4-7 μM, most preferably 4-6 μM CFSE.

Steps (e) and (f): Analysis of CTL Activity

The effector cells and target cells are mixed at a suitable ratio (with respect to cell number) and cultured to induce the lysis of target cells by effector cells.

The analysis of the lysis activity of effector cells may be carried out in a variety of processes depending on the type of labels on target cells. Preferably, where target cells are labeled with fluorescent substances (e.g., CFSE), FACS (fluorescence-activated cell sorter) is used to detect the lysis activity of effector cells.

The present method enables to measure more accurately and conveniently the activity of CTL of patients having various diseases or persons treated with various vaccines. For instance, where the present invention is intended to analyze the activity of CTL of AIDS patients or persons AIDS-vaccinated, it can be carried out using various HIV antigens (e.g., p24 and Nef) linked to the CTP.

In another aspect of this invention, there is provided a kit for measuring the activity of cytotoxic T lymphocytes (CTLs), comprising a cytoplasmic transduction peptide (CTP).

The present kit enables to measure more accurately and conveniently the activity of CTL of patients having various diseases or persons treated with various vaccines. In addition, the present kit allows for measuring the activity of CTL by use of primary cells instead of cell lines. Such striking feature is mainly due to the fact that the CTP delivers antigens into cytoplasm not nucleus, permitting target cells to present more effectively antigens on their surface through major histocompatibility complex class I (MHC I) molecules. The descriptions of CTP molecules are also found in WO 03/097671, the teachings of which are incorporated herein by reference.

As a main component, the CTP molecules permit primary cells to be used for generating target cells. In addition, they can be used in a process to maturate dendritic cells for providing effector cells. These novel applications of CTP molecules are firstly suggested by the present inventors.

According to a preferred embodiment, the CTP is a CTP-antigen complex in which the CTP is linked to an antigen molecule. Antigens linked to the CTP are any one derived from pathogenic microbes. For example, antigens include cancer-specific antigens or HIV antigens such as p24, Nef, env, Pol (reverse transcriptase), Tat, Pro (protease) and Int (integrase); hepatitis B virus (HBV) antigens such as Pre-S1, Pre-S2, S and core antigens; HCV antigens such as HCV core, E1 and E2; and human papilloma virus antigens such as L1, E1, E5, E6 and E7 antigens.

The length of the CTP used in this kit may vary within the acceptable scope of the art, preferably 9-20 amino acids, more preferably 9-15 amino acids, and most preferably, about 11 amino acids.

The principle strategy for designing the structure of the CTP peptide is that amino acid residues exhibiting α-helix stabilization or α-helix formation-enhancing properties as well as having positive-charged R-group are incorporated into the peptide, so that its binding affinity to importin-α is minimized and its transduction potential is improved or at least maintained.

The term “α-helix formation-enhancing amino acid” used herein refers to an amino acid having a strong tendency to form or stabilize α-helix conformation. The description of such a tendency is disclosed in W.H. Freeman's Proteins: Structure and Molecular Properties, p. 235 (1983). According to a preferred embodiment, the α-helix formation-enhancing amino acid essentially comprised in the peptide of this invention includes alanine, arginine and lysine; more preferably arginine and lysine; and most preferably, arginine.

The term “amino acid having a positive-charged R-group” used herein refers to a basic amino acid such as arginine, lysine and histidine, preferably arginine and lysine, and most preferably, arginine.

According to a preferred embodiment, the CTP of this invention comprises α-helix formation-enhancing amino acids having a positive-charged R-group as an essential amino acid. The term “an essential amino acid” used herein means that at least 3, preferably at least 5, more preferably at least 7, and most preferably at least 8 of α-helix formation-enhancing amino acids having a positive-charged R-group are essentially contained in the peptide of this invention.

According to a preferred embodiment, the peptide of this invention forms an α-helical structure and comprises at the site of its N-terminal, an amino acid residue which has a relatively high freedom at the φ and ψ rotations of the peptide unit. With respect to the rotations, “φ” refers to the rotation about the Cα-N single bond and “ψ” refers to rotation about Cα-C single bond. The amino acid residue which has a relatively high freedom at the φ and ψ rotations is glycine or alanine, or most preferably, glycine.

According to a specific example of this invention, the CTP of this invention comprises at least a peptide represented by the following formula: A-X₁-X₂-B-X₃-X₄-X₅-X₆-X₇-X₈. In this formula, A is an amino acid exhibiting relatively high freedom at the φ and ψ rotations of the peptide unit, and at least 3 of the residues of X₁, X₂, B, X₃, X₄, X₅, X₆, X₇, and X₈ are arginine or lysine.

According to a preferred embodiment, A is glycine or alanine, or more preferably, A is glycine. Preferably, at least 4, more preferably at least 5, still more preferably at least 6, and most preferably at least 7 of the residues of X₁, X₂, B, X₃, X₄, X₅, X₆, X₇, and X₈ are arginine or lysine, or preferably, arginine.

According to another specific example of this invention, the CTP of this invention comprises an amino acid sequence selected from the group consisting of SEQ ID NOs:1-14. Preferably, the peptide of this invention comprises an amino acid sequence selected from the group consisting of SEQ ID NOs:1-6, 8-10 and 13-14. More preferably, the peptide of this invention comprises an amino acid sequence selected from the group consisting of SEQ ID NOs:1-2 and 13-14. Still more preferably, the peptide of this invention comprises an amino acid sequence selected from the group consisting of SEQ ID NOs:1 and 13. Most preferably, the peptide of this invention comprises an amino acid sequence of SEQ ID NO:1.

According to a preferred embodiment, the kit comprise a recombinant fusion antigen prepared by linking HIV-1 p24 of SEQ ID NO:15, HIV-1 Nef of SEQ ID NO:16 or HCV core of SEQ ID NO:17 to the C-terminal of the CTP of SEQ ID NO:1.

The following specific examples are intended to be illustrative of the invention and should not be construed as limiting the scope of the invention as defined by appended claims.

### EXAMPLES

### Experimental Methods

Construction of Recombinant Plasmids Expressing CTP-p24, CTP-Nef, CTP-HCVcore or CTP-Albumin

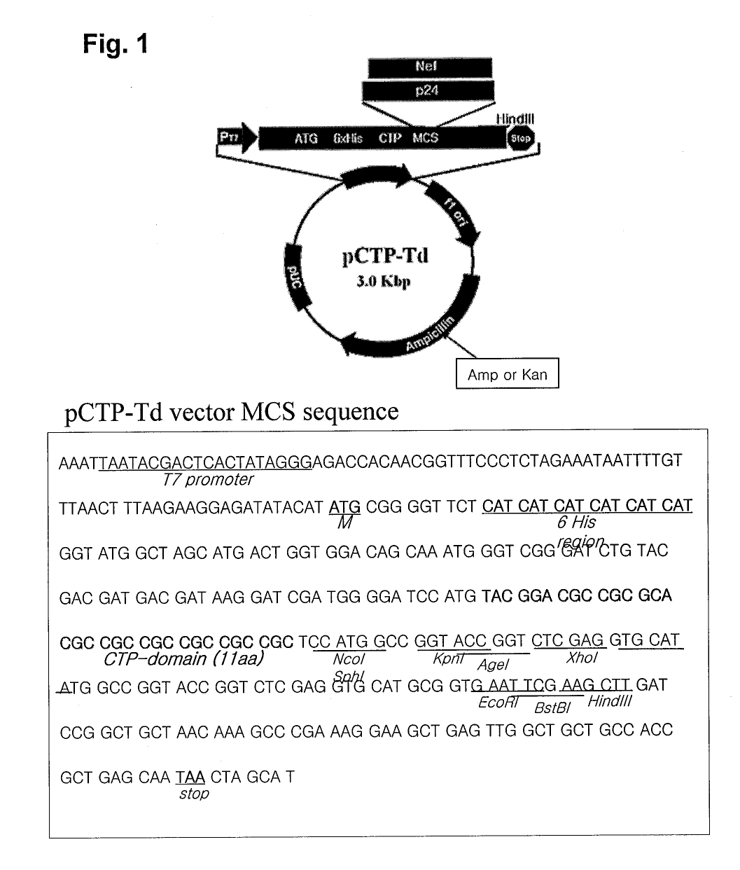

The pCTP-Td vector (see FIG. 1) was prepared as follows: The pCTP-Td vector was produced by genetically manipulating the pTAT-HA vector (kindly provided by Dr. Dowdy at Washington University School of Medicine, H. Nagahara et al., Nature Med. 4:1449 (1998)). The pTAT-HA vector was digested with BamHI and NcoI for 2 hr at 37° C. to remove the Tat domain and HA-tag region, and purified by a gel extraction kit (Nucleogen). The complementary forward (5′-GAT CCA TGT ACG GAC GCC GCG CAC GCC GCC GCC GCC GCC GCT C-3′) and reverse primers (5′-CAT GGA GCG GCG GCG GCG GCG GCG TGC GCG GCG TCC GTA CAT G-3′) were synthesized (Bionics, Inc.) to obtain a DNA fragment encoding the CTP (nucleotide sequence, TAC GGA CGC CGC GCA CGC CGC CGC CGC CGC CGC; amino acid sequence, YGRRARRRRRR). The primers were heated for 5 min at 95° C. and cooled to room temperature at a rate of 1° C./min to give a double stranded DNA molecule. The primers were designed to ensure the final CTP DNA fragment to have cohesive ends for BamHI and NcoI. The annealed CTP DNA fragments were purified by a PCR purification kit (Nucleogen) and ligated into the vector for 18 hr at 16° C. using T4 DNA ligase (Roche) followed by transforming into E. coli JM109 (Stratagene) to produce a recombinant plasmid, pCTP-Td. The construction of the CTP-Td vector was verified by digestion with BamHI and NcoI and sequencing (SolGent, Inc).

Then, p24 and Nef genes were PCR-amplified using cDNA from HXBc2 clone of HIV-1 clones and the following primer sets: HIV-p24, 5′-TGC CCT CTC GAG CCT ATA GTG CAG AAC ATC-3′ (forward primer) and 5′-CTA CAG AAT TCG CCA AAA CTC TTG CCT TAT-3′ (reverse primer); HIV-nef, 5′-GGG GTC GAC ATG GGA GGC AAG TGG TCA AAA-3′ (forward primer) and 5′-GGG GAA TTC TCA GTT CTT GAA GTA CTC CGG-3′ (reverse primer). The PCR-amplified products were cloned into XhoI and EcoRI sites of the pCTP-Td vector. The expression constructs obtained thus carry the nucleotide sequences of the following amino acid sequences: His-tag-CTP (YGRRARRRRRR)-p24 or -Nef. The amino acid sequences of inserts, HIV-1 p24 and Nef, can be found in SEQ ID NOs:15 and 16.

Finally, the pCTP-Td vector was digested with BamHI and XhoI to obtain HIV-1 p24 or Nef encoding DNA fragments and the DNA fragments were then inserted into pET28a⁺ (NOVAGENE, Inc.), which makes it more feasible to massively produce recombinant proteins in medium containing kanamycin.

Unlike p24 and Nef proteins, the HCV core protein could not be expressed using conventional vectors. For this reason, we designed to express the HCV core protein in the form of fusion protein in which the HCV core protein was linked to the C-terminal of the Nus protein of pET43.1 (NOVAGENE, Inc.). The Nus protein permitted to elevate the solubility of the recombinant protein, the HCV core protein. The nucleotide sequence of the HCV core was PCR-amplified by using the E1E2 gene as templates (kindly provided by Dr. Young-Chul SUNG at POHANG University of Science and Technology) and the following primer set: 5′-cat gaa ttc tac gga cgc cgc gca cgc cgc cgc cgc cgc cgc atg agc aca aat cct aaa-3′ (forward primer); and 5′-ccg ctc gag tta agc aga aac tgg ggt-3′ (reverse primer). The PCR-amplified products were cloned into XhoI and EcoRI sites of the pET43.1 vector. The expression constructs obtained thus carry the nucleotide sequences of the following amino acid sequences: Nus-CTP(YGRRARRRRRR)-HCVcore.

For producing the CTP-Albumin (human serum albumin) as a control, total RNAs were extracted from HepG2 using Trizol reagent (Invitrogen, Inc.) and employed for the synthesis of cDNAs using oligo-dT primer, followed by PCR-amplification using the following primer set: 5′-CAT GAA TTC TAC GGA CGC CGC GCA CGC CGC CGC CGC CGC CGC ACT ATG GCT GAT GAG TCA GCT GAA-3′ (forward primer); 5′-CCG CTC GAG CTA TTA ATC AGG ATG CCT TCT TGC A-3′ (reverse primer). The PCR amplicons were cloned into XhoI and EcoRI sites of the pET28a vector.

Production and Purification of Recombinant Proteins, CTP-p24, CTP-Nef and CTP-HCVcore

E. coli BL21Gold (Stratagene) was transformed with recombinant plasmids carrying HIV-1 p24 or Nef genes to prepare transformants according to Hanahan method. The transformed microbes were inoculated into 100 μg/ml of ampicillin-LB medium and cultured for 20 hr at 37° C. The transformants cultured in the LB medium were harvested and suspended in lysis buffer (50 mM NaH₂PO₄, 300 mM NaCl, 10 mM imidazole, pH 8.0), lysozime (1 mg/ml) and 1 mM PMSF. The suspension was subjected to sonication to yield unpurified protein solution. The soluble aliquots were passed twice through 3 ml of Ni⁺-NTA resin (Qiagen) and then the resin was washed twice with 15 ml of washing buffer (50 mM NaH₂PO₄, 300 mM NaCl, 20 mM imidazole, pH 8.0). The elution was carried out by use of elution buffer (50 mM NaH₂PO₄, 300 mM NaCl, 250 mM imidazole, pH 8.0).

The analysis on SDS-PAGE was performed to verify the purity of proteins. The eluted protein solution underwent dialysis for more than 12 hr against 5 L phosphate buffer. The concentration of the protein solution was determined using Bradford solution, aliquoted into tubes and stored at −20° C.

The CTP-HCVcore was also expressed and purified in the same manner as CTP-p24, except that 2×YT medium was used instead of LB medium.

Transduction Efficiency of Recombinant Proteins, CTP-p24, CTP-Nef and CTP-HCVcore

To elucidate the transduction potential of recombinant proteins, each of CTP-p24 and CTP-Nef was dissolved in 1 ml of distilled water and incubated for 20 hr at a concentration of 100 μg/ml with dendritic cells. Mature dendritic cells were used for minimizing the proteolysis of transported proteins. 5-7.5×10⁶ cells/ml of PBMCs were suspended in RPMI 1640 medium supplemented with 2% human serum and aliquoted into 12-well plates, followed by incubating for 2 hr in CO₂ incubator (37° C., 5% CO₂) to adhere monocytes to plates. The plates were well shaken to remove non-adherent cells and washed three times with pre-warmed (at 37° C.) RPMI 1640 medium. The medium for dendritic cells (Mo-DC) was prepared by adding 0.5 μg/ml of hIL-4 (Endogene, Inc.) and 100 ng/ml of hGM-CSF (LG, Inc.) into X-VIVO 15 medium and its 1 ml-aliquot was added to each well of the plates, after which culture was carried out. After 3-day culture, the medium was replaced with 0.5 ml of a fresh medium for dendritic cells. On 5 days of culture, non-adherent cells (immature dendritic cells) were harvested and suspended in the DC maturation medium [X-VIVO medium supplemented with 10 ng/ml of IL-6 (100 U/ml, Endogen), 10 ng/ml of IL-1b (Endogen), 10 ng/ml of TNF-α (Endogen), 1 μg/ml of PGE2 (SIGMA) and 1000 U/ml of IFN-γ (LG)], followed by culturing. On 7 days of culture, mature dendritic cells were harvested. The dendritic cells were treated with each of CTP-p24 and CTP-Nef for 20 hr, trypsinized for 90 sec to remove residual proteins on the surface of dendritic cells and washed three times with PBS. The cells were disrupted using a sonicator and lysates were resolved on 12% SDS-PAGE. Then, the Western blotting was performed using monoclonal antibodies (MAB880-A, CHEMICON, Inc.) to verify cell-entry of antigens. For the Western blotting to verify the transduction of CTP-HCVcore, MA1-080 (HCVcore mouse monoclonal antibody IgG₁, Affinity BioRegent Inc.) and anti-mouse AP-conjugated antibody (Sigma Chem. Inc.) were used as primary and secondary antibodies, respectively. Dot blotting was carried out using the same antibodies as described above so as to quantitate the antigens introduced into cells.

Collection and Storage of Blood

20 ml of blood from each subject for the activity of CTL were collected and then diluted with PBS, if necessary. The blood samples were centrifuged with density gradient medium such as ficol-paque solution or Lymphoprep™ to obtain peripheral blood mononuclear cells, PBMCs. The cells obtained thus were aliquoted (1.0-2.0×10⁷ cells/vial) and freezing-stored for producing dendritic cells, effector and target cells, except those for preparing initial dendritic cells.

Preparing Monocyte-Derived Dendritic Cells (MoDC) from PBMCs

PBMCs were suspended in RPMI 1640 medium supplemented with 2% human serum to a density of 5-7.5×10⁶ cells/ml, and 1 ml of the cell suspension was aliquoted into each well of 12-well plates, followed by incubating for 2 hr in a CO₂ incubator (37° C., 5% CO₂) to adhere monocytes to plates. The plates were well shaken to remove non-adherent cells and washed three times with pre-warmed (at 37° C.) RPMI 1640 medium. The medium for dendritic cells (Mo-DC) was prepared by adding 0.5 μg/ml of hIL-4 (Endogene, Inc.) and 100 ng/ml of hGM-CSF (LG, Inc.) into X-VIVO 15 medium and its 1 ml-aliquot was added to each well of the plates, after which culture was carried out. After 3-day culture, the medium was replaced with 0.5 ml of a fresh medium for dendritic cells. On 5 days of culture, the cells were incubated for 20 hr with 100 μg/ml of CTP-p24 or CTP-Nef, to which 10 ng/ml of IL-6 (100 U/ml, Endogen), 10 ng/ml of IL-1b (Endogen), 10 ng/ml of TNF-α (Endogen), 1 μg/ml of PGE2 (SIGMA) and 1000 U/ml of IFN-γ (LG)] as cytokines for DC maturation medium were added. On 7 days of culture, mature dendritic cells were harvested and used as antigen presenting cells.

The cell surface phenotype of dendritic cells subject to maturation culture was analyzed by FACS (fluorescence-activated cell sorter). The dendritic cells were incubated with FITC (fluorescein isothiocyanate) and PE (R-phycoerythrin)-conjugated antibodies (CD1a, CD40, CD80, CD83, CD86, HLA-D and HLA-A, B, C antibodies, Pharmingen) for 20 min at 4° C. Then, cells were washed with FACS buffer (0.5% BSA, 0.1% sodium azide in PBS) and their surface antigens were analyzed using FACSCalibur and Cell Quest software (Becton Dickison, USA).

Generation of Effector Cell (CTL) from Non-Adherent PBMCs

Non-adherent cells obtained and stored during the preparation of MoDC described above were thawed (considering cell death and loss in preparation of lymphocytes, at least 4×10⁶ cells were initially used). The mature antigen presenting cells (mature APCs) suspended in X-VIVO 15 medium and progenitor cells for preparing effector cells (pre-effector cells) were mixed at a ratio of 1:10. 2 ml of pre-effector cells (1×10⁶ cells/ml) were aliquoted to a 6-well plate and cultured for 7 days in a CO₂ incubator (37° C.) without exchanging medium. Then, 2 ml of antigen presenting dendritic cells suspended in X-VIVO 15 medium (1×10⁶ cells/ml) were aliquoted to the 6-well plate and cultured additionally for 7 days.

CTLs prepared and amplified for 2 weeks using DC were collected in a 15 ml tube and ½ volume of Lymphoprep™ (density 1.077, Axis Shield Inc.) was applied to the tube, followed by centrifugation under 2,000 rpm (800×g) for 25 min at room temperature, resulting in the selective isolation of live cells. The cells positioned between sample and medium were collected, washed twice with RPMI-1640 medium and counted. Finally, the concentration of the cell suspension was adjusted with X-VIVO 15 medium to 2×10⁶ cells/ml.

Generation of Target Cells: Antigen Pulsing and Labeling with Fluorescent Substance

1.2×10⁶ cells/ml of fresh PBMCs or PBMCs stimulated overnight with IFN-γ (1000 U/ml) or Con-A (1 μg/ml) were aliquoted into a 6-well plate and incubated for 19-20 hr with 50 μg/ml of CTP-p24 or control antigen (CTP-β-galactosidase) in X-VIVO 15 medium. After harvesting cells, they were washed once with fresh RPMI 1640 medium. A stock solution of CFSE (carboxyfluorescein diacetate succinimidyl ester, Vybrant™ CFDA SE Cell Tracer Kit, Molecular Probes Inc.) was prepared to a concentration of 10 mM, after which 4 μl of the solution was diluted by a factor of 10 with X-VIVO 15 medium (the final conc. 1 mM). The cells were well suspended in 500 μl of X-VIVO 15 medium. The antigen-treated groups (CTP-p24 or CTP-β-gal) were incubated for 15 min at 37° C. with 8 μl of CFSE (1 mM), which corresponds to CFSE^{high} cells (the final conc. of CFSE to 16 μM), and the antigen-untreated group with 8 μl of CFSE for 15 min at 37° C., which corresponds to CFSE^{low} cells (the final conc. of CFSE to 4 μM), leading to CFSE labeling on cell surface. Then, the cells were washed once with RPMI 1640 medium and suspended in 1 ml of X-VIVO 15 medium, followed by incubating for 30 min at 37° C. The cells were finally washed with X-VIVO 15 medium and their number was exactly counted. The concentration of the cell suspension was adjusted with X-VIVO 15 medium to 2×10⁶ cells/ml. The same amounts of antigen-treated and antigen-untreated groups were mixed.

Measurement of the CTL Activity

The effector and target cells exactly counted were mixed at ratios of 0.5:1, 1:1, 2:1 and 4:1, and incubated in 5 ml polystyrene tube (BD Inc.). The tube was subject to centrifugation under 500 rpm for 2 min to promote the reaction between cells and then the cells in X-VIVO 15 medium were incubated for 4-5 hr at 37° C. in a CO₂ incubator. Thereafter, dead cells in labeled cells were analyzed by FACS (fluorescence-activated cell sorter) using FACSCalibur and Cell Quest software (Becton Dickinson, USA). The event count of collection criteria of FACS was adjusted to 250,000 cells. Where CFSE-labeled target cells were counted constantly to 5×10³ cells, the FACS measurement was stopped and data were stored. The CTL activity was calculated in accordance with the following mathematical function: ratio=% CFSE^{low}/% CFSE^{high}; percentage specific lysis activity=[1−(ratio of reactions containing solely target cells/ratio of reactions containing both effector and target cells)]×100.

Results: Production and Purification of Recombinant Proteins, CTP-p24, CTP-Nef and CTP-HCVcore

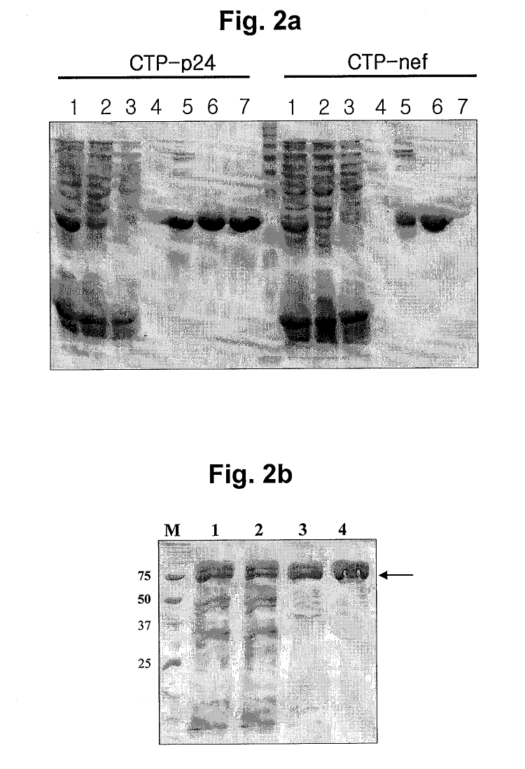

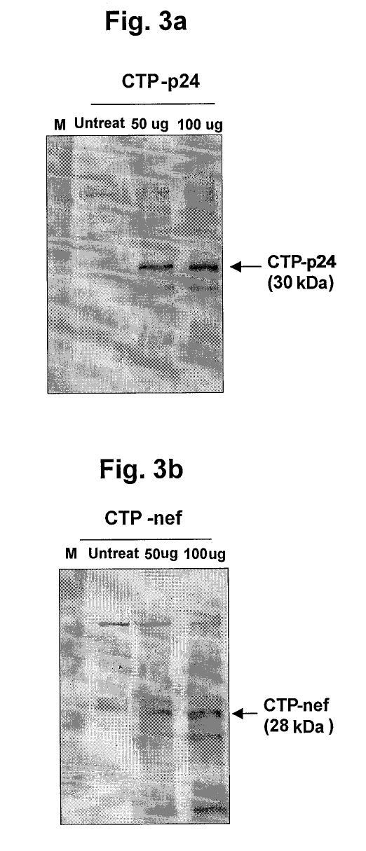

The recombinant proteins expressed and purified from transformants in 250 ml LB medium were quantitated to be about 10-20 mg. As shown in FIGS. 2a and 2b, the recombinant proteins, CTP-p24, CTP-Nef and CTP-HCVcore were purified.

Transduction Efficiency of Recombinant Proteins, CTP-p24, CTP-Nef and CTP-HCVcore

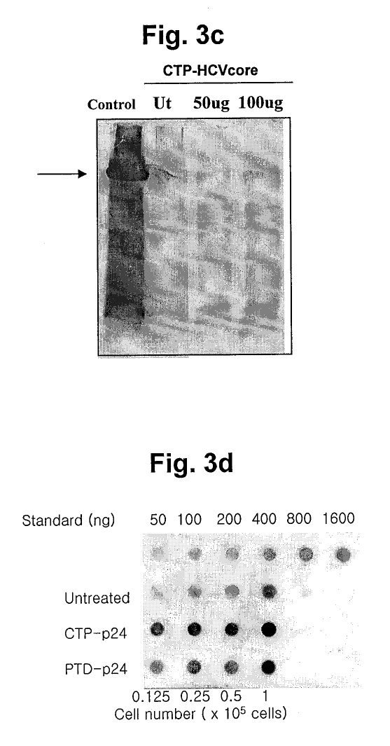

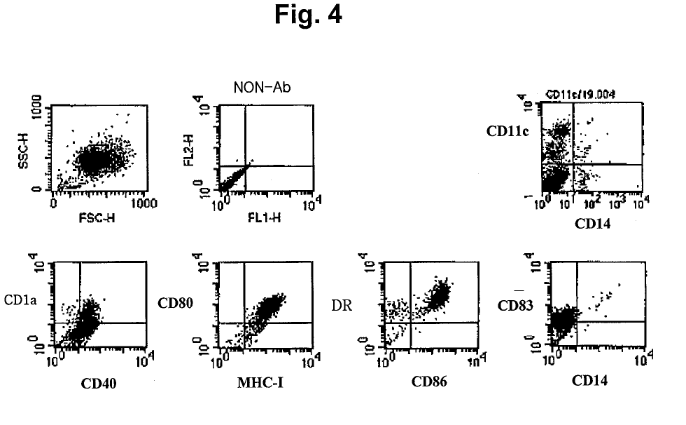

To reveal the transduction potential of CTP-linked recombinant proteins, dendritic cells were incubated for 20 hr with 50 or 100 μg/ml of purified recombinant proteins, harvested and subject to the Western blotting by use of specific antibodies. The results show that recombinant proteins were introduced into cells in a dose dependent manner (see FIGS. 3a-3c). For quantitating proteins introduced into cells, dendritic cells incubated for 20 hr with 100 μg/ml of CTP-p24 were analyzed by the dot blotting. The results demonstrate that at least 1 ng of CTP-linked proteins were shown to be introduced into each cell (FIG. 3c).

Preparing Dendritic Cells from PBMCs

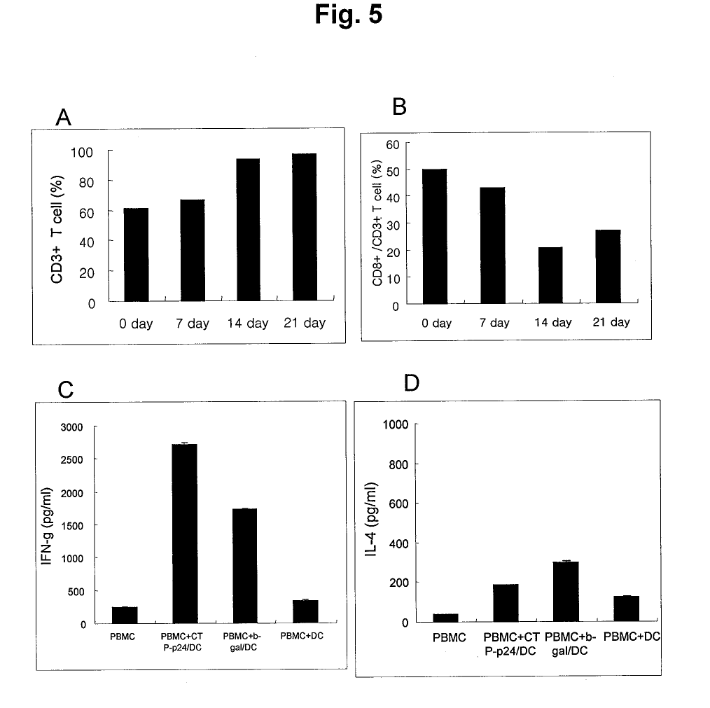

The maturation of dendritic cells was analyzed by FACS using CD1a, CD40, CD80, CD83, CD86, HLA-D and HLA-A, B, C antibodies. As represented in FIG. 4, it was verified that mature dendritic cells were generated without contamination of other cell types such as monocyte. The higher level of costimulatory factors, CD80, CD83, CD86 on DC surface shows clearly the maturation of DC.

Generation of Antigen-Specific Effector Cells

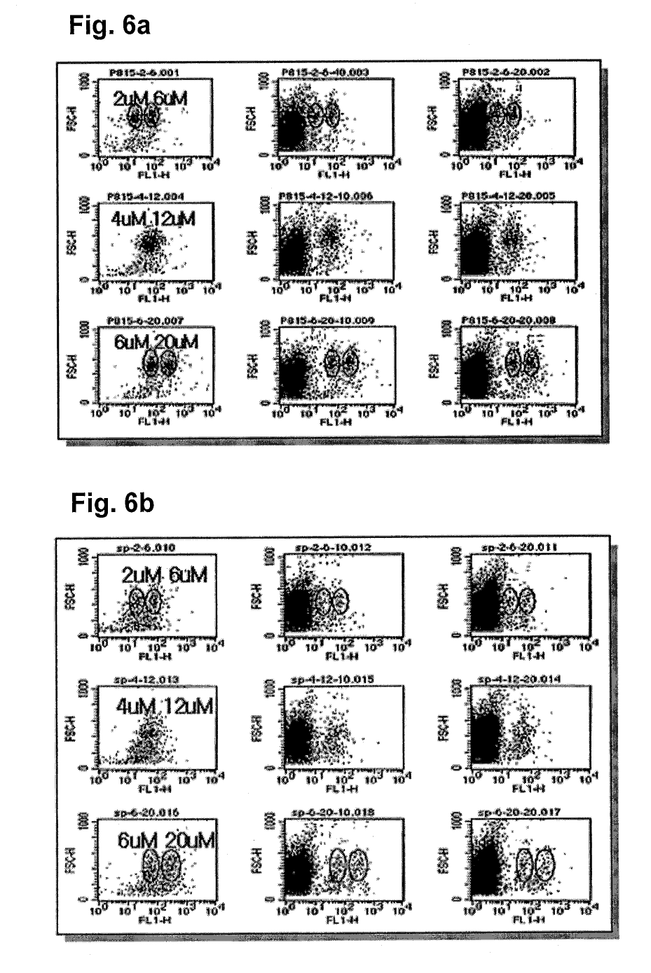

Non-adherent cells obtained from PBMCs of healthy person were mixed and cocultured for 20 days with CTP-p24-pulsed dendritic cells at a ratio of 10:1, giving rise to the amplification of effector cells (CTL). T cells stimulated and amplified with dendritic cells were analyzed for revealing the ratio of CD8⁺/CD3⁺, which demonstrates the generation of CTL. As represented in panels A and B of FIG. 5, while the number of CD3⁺ T cells increased, that of CD8⁺ T cells comprising CTL showed a decreasing pattern up to day 14 but a increasing pattern on day 21. Based on these results, it could be understood that p24-specific CD8⁺ T cells initially were initially present in a low level, in turn amplified by p24-pulsed dendritic cells to become a prominent cell type in total CD8⁺ T cells, and non-specific CD8⁺ T cells were decreased. In other words, PBMCs were stimulated for 21 days with CTP-p24-pulsed dendritic cells to amplify p24-specific CTL, giving rise to the increase in the population of T cells capable of recognizing p24 antigen. Following the 21-day stimulation, about 97% of cells were differentiated into T cells.

The differentiated CD8⁺ T cells were indirectly verified to posses the CTL activity by measuring the generation of γ-IFN (FIG. 5, panel C). Monocytes treated with the control antigen (β-gal) secreted γ-IFN at a level of 50% compared to DC. This result was presumed to result from the fact that monocytes treated capture antigens through phagocytosis and then stimulate T cells. In FIG. 5, panel D, IL-4 was shown to be secreted in a much lower level than γ-IFN, demonstrating that p24-specific T cells stimulated by dendritic cells were induced through Th1 immune reaction relating directly to CTL induction.

Establishment of CFSE Labeling Method to Target Cell

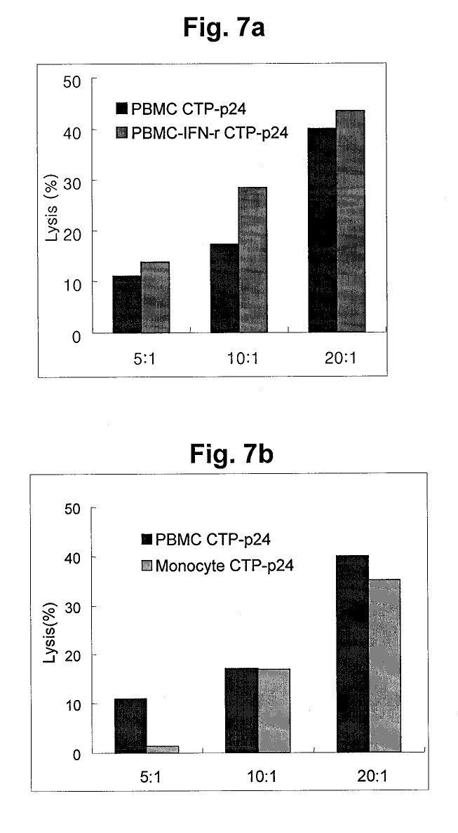

The spleen was removed by abdominal incision of 6-week-old C57BL/6 mice purchased from Samtako Inc. (Korea), grounded using a nylon mesh, removed of red blood cells using a lysis buffer and washed twice with RPMI-1640 medium to prepare mouse splenocytes. The mouse splenocytes and P815 cell line (Korean Cell Line Bank) not pulsed with antigens were labeled with each of 2 μM, 4 μM and 6 μM CFSE. The target cells pulsed with CTP-p24 antigen was also labeled with 6 μM, 12 μM and 20 μM CFSE in the same manner. The target cells pulsed and not-pulsed with antigens were mixed in the same number, and then mixed with 10-fold or 20-fold number of unlabeled splenocytes. CFSE-labeled target cells were examined to be distinctly detected in FACS analysis. As shown in FIG. 6a, for CFSE-labeled P815 cells, 2 μM and 6 μM CFSE staining of control and antigen-pulsed target cells showed overlapped results, making it impossible to accurately analyze. Also, 4 μM and 12 μM CFSE staining resulted in overlapped gates. In contrast, target cells stained with 6 μM and 20 μM CFSE was shown to be no overlapped results with unlabeled splenocytes and to be distinctly differentiable results between target cells. In addition, 6 μM and 20 μM CFSE staining for primary splenocytes showed distinctly differentiable results between target cells as shown in FIG. 6b. Based on these results, we established the CFSE labeling strategy for detecting more clearly the death of target cells in which antigen-pulsed target cells are labeled with 20 μM CFSE and control target cells not pulsed are labeled with 6 μM CFSE, enabling the lysis of target cells by CTL to be analyzed in a more accurate manner.

Generation of Target Cells by Pulsing PBMCs with CTP-p24

To generate target cells susceptible to the lysis activity of CTL, target cells were pulsed in accordance with various conditions. PBMCs pre-incubated with 1000 U/ml of γ-IFN for 7 hr, or PBMCs not incubated were pulsed for 20 hr with 50 μg/ml of CTP-p24 antigen. Then, antigen-pulsed target cells were labeled with 20 μM CFSE and control target cells not pulsed were labeled with 4-6 μM CFSE for 15 min at 37° C.

Measurement of CTL Activity against p24 CTL Activity Using 2×10⁵ cells/ml or 2×10⁶ cells/ml of Target Cell

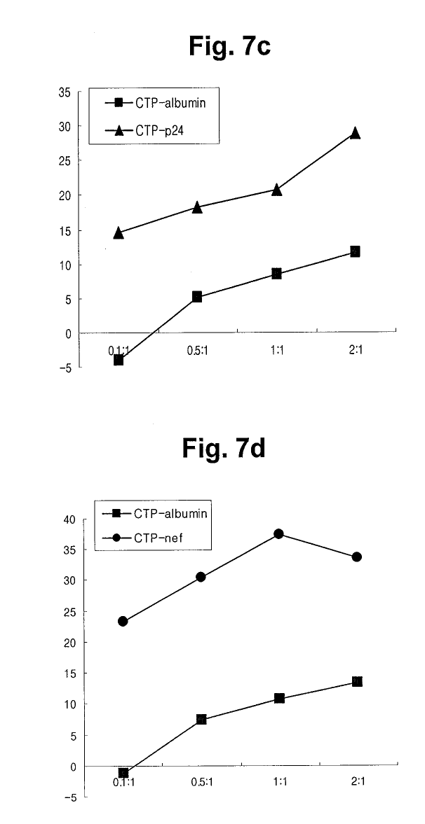

The effector cells and target cells (2×10⁵ cells/ml or 2×10⁶ cells/ml) were mixed at ratios of 5:1, 10:1 and 20:1, centrifuged under 500 rpm for 2 min and incubated for 4-5 hr at 37° C. in a CO₂ incubator. Finally, the lysis of target cells was analyzed by FACS method as described previously. As represented in FIG. 7a, CTLs lyzed the target cells pre-treated with γ-IFN in a more sensitive manner than untreated PBMCs as target cells. Where monocytes prepared by using MACS (Miletenyi Biotech Inc.) were used as target cells, the linearity (reliability) of the CTL responses was slightly improved (FIG. 7b). To elucidate the specificity of CTL, the activity of CTL was measured using as target cells PBMCs pulsed with CTP-β-galactosidase or CTP-p24. FIG. 7c clearly shows that CTLs generated exhibit high specificity against p24. To determine whether activities observed in the measurements of CTL activity are antigen-specific, the CTL activity was measured using as target cells PBMMCs pulsed with each of CTP-albumin (human serum protein), CTP-p24 and CTP-Nef. As results, it was revealed that CTLs induced against p24 and Nef exhibit high specificity to target cells pulsed with CTP-p24 and CTP-Nef, respectively. In contrast, CTLs induced against p24 and Nef were shown to have much lower activity to target cells pulsed with CTP-albumin.

Most Effective Measurement of CTL Activity Using Counts of PBMCs 10-Fold Greater Than Conventional Counts as Target Cells

To elevate the linearity of CTL reactions, CTL reactions were carried out using counts of PBMCs 10-fold greater than conventional counts.

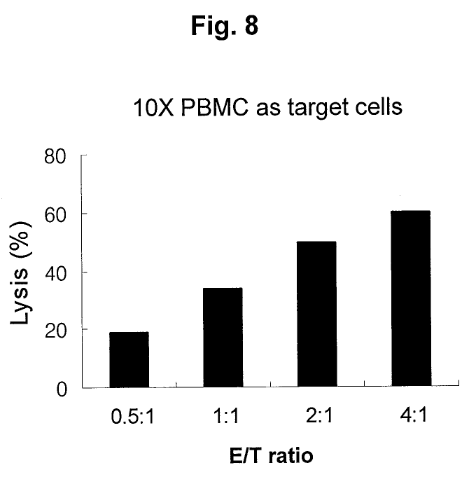

The reactions of effector and target cells were adjusted to ratios of 0.5:1, 1:1, 2:1 and 4:1 by varying the number of effector cells with a fixed number of target cells (1×10⁵ cells/well). The cell mixtures were centrifuged under 500 rpm for 2 min and incubated for 4.5 hr at 37° C. in a CO₂ incubator, after which the lysis of target cells was analyzed by FACS. As represented in FIG. 8, where the number of target cells increased by ten-fold, the CTL reactions were found to be more linear and effective than any conventional. In addition, it was shown that the linearity of CTL reactions reflects the ratio of E/T.

The present invention ensures to prepare target cells more feasibly and to elevate the linearity of CTL reactions, leading to the most effective measurement of CTL activity.

As described previously, the present invention provides a novel method for measuring the activity of cytotoxic T lymphocytes (CTLs) and a kit therefor. The present invention ensures target cells to be prepared by use of primary cells without establishing cell lines. In addition, the present invention enables to measure the CTL activity more accurately and conveniently without using radioisotopes. Accordingly, it could be understood that the present invention is considerably useful in determining whether the immune responses are effectively induced upon vaccination, ensuring to provide a promising approach for much more reliable and convenient prognosis of diseases.

Having described preferred embodiments of the present invention, it is to be understood that variants and modifications thereof falling within the spirit of the invention may become apparent to those skilled in this art, and the scope of this invention is to be determined by appended claims and their equivalents.

What is claimed is:

1. A method for measuring the activity of cytotoxic T lymphocytes (CTLs), which comprises the steps of:
(a) preparing peripheral blood mononuclear cells (PBMCs) from blood of an animal;
(b) preparing mature dendritic cells by isolating monocytes from the PBMCs, differentiating the monocytes into dendritic cells for presenting an antigen molecule and pulsing dendritic cells with the antigen molecule to obtain the mature dendritic cells;
(c) preparing the CTLs as an effector cell by stimulating the PBMCs with the mature dendritic cells to activate and amplify the CTLs;
(d) preparing target cells by pulsing the PBMCs, monocytes or B cells with a cytoplasmic transduction peptide (CTP)-antigen complex generated by linking the antigen molecule of step (b) to the CTP;
(e) treating the target cells with the effector cells; and
(f) analyzing the lysis of the target cells.

2. The method according to claim 1, wherein the antigen molecule for preparing the mature dendritic cells is linked to the CTP.

3. The method according to claim 1, wherein the target cells are PBMC.

4. The method according to claim 3, wherein the PBMC is a primary cell.

5. The method according to claim 1, wherein the PBMCs are isolated from human or out-bred animals.

6. The method according to claim 1, wherein the CTP comprises a peptide represented by the following formula:
A-X₁-X₂-B-X₃-X₄-X₅-X₆-X₇-X₈
wherein A is an amino acid exhibiting relatively high freedom at the φ and ψ rotations of a peptide unit, and at least 3 residues of X₁, X₂, B, X₃, X₄, X₅, X₆, X₇, and X₈ are arginine or lysine.

7. The method according to claim 6, wherein the A is glycine or alanine.

8. The method according to claim 6, wherein at least 4 residues of X₁, X₂, B, X₃, X₄, X₅, X₆, X₇, and X₈ are arginine or lysine.

9. The method according to claim 9, wherein the CTP comprises an amino acid sequence selected from the group consisting of SEQ ID NOs:1-14.

10. The method according to claim 9, wherein the CTP comprises an amino acid sequence selected from the group consisting of SEQ ID NOs:1 and 13.

11. A kit for measuring the activity of cytotoxic T lymphocytes (CTLs), comprising a cytoplasmic transduction peptide (CTP).

12. The kit according to claim 11, wherein the CTP comprises α-helix formation-enhancing amino acids having a positively-charged R-group as an essential amino acid.

13. The kit according to claim 11, wherein the CTP comprises at or near the N-terminal of its α-helix region an amino acid exhibiting relatively high freedom at the φ and ψ rotations of a peptide unit.

14. The kit according to claim 12, wherein the amino acid is arginine or lysine.

15. The kit according to claim 14, wherein the amino acid is arginine.

16. The kit according to claim 11, wherein the CTP comprises a peptide represented by the following formula:
A-X₁-X₂-B-X₃-X₄-X₅-X₆-X₇-X₈
wherein A is an amino acid exhibiting relatively high freedom at the φ and ψ rotations of a peptide unit, and at least 3 residues of X₁, X₂, B, X₃, X₄, X₅, X₆, X₇, and X₈ are arginine or lysine.

17. The kit according to claim 16, wherein the A is glycine or alanine.

18. The kit according to claim 17, wherein the A is glycine.

19. The kit according to claim 16, wherein at least 4 residues of X₁, X₂, B, X₃, X₄, X₅, X₆, X₇, and X₈ are arginine or lysine.

20. The kit according to claim 19, wherein at least 5 residues of X₁, X₂, B, X₃, X₄, X₅, X₆, X₇, and X₈ are arginine or lysine.

21. The kit according to claim 20, wherein at least 6 residues of X₁, X₂, B, X₃, X₄, X₅, X₆, X₇, and X₈ are arginine or lysine.

22. The kit according to claim 21, wherein at least 7 residues of X₁, X₂, B, X₃, X₄, X₅, X₆, X₇, and X₈ are arginine or lysine.

23. The kit according to claim 11, wherein the CTP comprises an amino acid sequence selected from the group consisting of SEQ ID NOs:1-14.

24. The kit according to claim 23, wherein the CTP comprises an amino acid sequence selected from the group consisting of SEQ ID NOs:1-6, 8-10 and 13-14.

25. The kit according to claim 24, wherein the CTP comprises an amino acid sequence selected from the group consisting of SEQ ID NOs:1 and 13.

26. The kit according to claim 11, wherein the CTP is linked to an antigen molecule.

27. The kit according to claim 11, wherein the CTP is CTP-p24 or CTP-Nef prepared by linking p24 or Nef of HIV (human immunodeficiency virus) to the CTP, or CTP-HCVcore prepared by linking core of HCV (hepatitis C virus) to the CTP; and the kit is used for measuring the activity of CTLs against AIDS or hepatitis C.


SEQUENCE LISTING (17 sequences)

SEQ ID NO: 1 - protein, 11 aa, Artificial Sequence
  cytoplasmic transduction peptide
YGRRARRRRRR

SEQ ID NO: 2 - protein, 11 aa, Artificial Sequence
  cytoplasmic transduction peptide
YGRRARRRARR

SEQ ID NO: 3 - protein, 11 aa, Artificial Sequence
  cytoplasmic transduction peptide
YGRRARRAARR

SEQ ID NO: 4 - protein, 11 aa, Artificial Sequence
  cytoplasmic transduction peptide
YKRKARRAARR

SEQ ID NO: 5 - protein, 11 aa, Artificial Sequence
  cytoplasmic transduction peptide
YARKARRAARR

SEQ ID NO: 6 - protein, 11 aa, Artificial Sequence
  cytoplasmic transduction peptide
YKRAARRAARR

SEQ ID NO: 7 - protein, 11 aa, Artificial Sequence
  cytoplasmic transduction peptide
YEREARRAARR

SEQ ID NO: 8 - protein, 11 aa, Artificial Sequence
  cytoplasmic transduction peptide
YAREARRAARR

SEQ ID NO: 9 - protein, 11 aa, Artificial Sequence
  cytoplasmic transduction peptide
YGRAARRAARR

SEQ ID NO: 10 - protein, 11 aa, Artificial Sequence
  cytoplasmic transduction peptide
YRRAARRAARA

SEQ ID NO: 11 - protein, 11 aa, Artificial Sequence
  cytoplasmic transduction peptide
YPRAARRAARR

SEQ ID NO: 12 - protein, 11 aa, Artificial Sequence
  cytoplasmic transduction peptide
PARAARRAARR

SEQ ID NO: 13 - protein, 11 aa, Artificial Sequence
  cytoplasmic transduction peptide
YGRRRRRRRRR

SEQ ID NO: 14 - protein, 11 aa, Artificial Sequence
  cytoplasmic transduction peptide
YRRRRRRRRRR

SEQ ID NO: 15 - protein, 231 aa, Human immunodeficiency virus type 1
PIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 16 - protein, 204 aa, Human immunodeficiency virus type 1
MGGKWSKRRAEGWQTIRERMRRAEPAEPAADGVGAVSRDLARHGAITSSNTNNADIAWLEAQEEGEVGFPVRPQVPLRPMTYKAAVDLSHFLKEKGGLEGLVHSQKRQDILDLWVYHTQGFFPDWQNYTPGPGIRYPLTFGWCYKLVPVEPDEGENNREDNSLLHPANQHGVEDSERQVLVWRFDSRLAFHHVARELHPEYFKN

SEQ ID NO: 17 - protein, 191 aa, Hepatitis C virus
MSTNPKPQRQTKRNTNRRPKDIKFPGGGQIVGGVYLLPRRGPRLGVRAARKASERSEPRSKRQRIPKARQPTGRHWGQPGYPWPLYGNEGCGWAGWLLSPRGSRPSWGPNDPRRRSRNLGKVIDTLTCGLADLFGYIPVVGGPLGGVAAALAHGVRAVEDGINYATGNLPGCSFSIFLLALLSCLTVPASA